(19) United States
(12) Patent Application Publication
(10) Pub. No.: US 2015/0128353 A1
(43) Pub. Date: May 14, 2015

(54) SLEEP MONITORING SYSTEM AND METHOD

(71) Applicant: Boyd Thomas KILDEY, Brisbane (AU)

(72) Inventor: Boyd Thomas KILDEY, Brisbane (AU)

(21) Appl. No.: 14/601,620

(22) Filed: Jan. 21, 2015

Related U.S. Application Data
(63) Continuation-in-part of application No. 13/970,354, filed on Aug. 19, 2013.
(60) Provisional application No. 61/699,267, filed on Sep. 10, 2012.

(30) Foreign Application Priority Data
Sep. 11, 2012 (AU) 2012903949
Aug. 14, 2013 (AU) 2013216618
Mar. 13, 2014 (AU) 2014201473

(51) Int. Cl. A61B 5/00; A61B 5/0205; A61B 5/11; A47C 27/08

(52) U.S. Cl. CPC A61B 5/4812; A47C 27/082; A47C 27/083; A61B 5/02055; A61B 5/11; A61B 5/742; A61B 5/024
USPC 5/706; 5/690; 600/300; 600/301; 600/508; 600/549; 600/529; 600/595

(57) ABSTRACT

An improved method and system for monitoring a user's sleep is provided. The method includes monitoring, using one or more sensors of a bed, biological signal data of a user; determining, by a processor, a sleep phase of the user according to the biological signal data; and providing, on a data interface, a sleep cycle report, determined at least partially according to the sleep phase of the user.

### TECHNICAL FIELD

The present invention relates to sleep monitoring, and in particular, although not exclusively, to sleep monitoring using sensors of a bed.

### BACKGROUND ART

Sleep monitoring has been extensively used to evaluate medical conditions, such as insomnia and sleep disorders. More recently, however, sleep monitoring has also been used in a non-medical context to determine a quality of a user's sleep with a view of improving the quality of the user's sleep.

Sleep diaries have been used to monitor a user's sleep, and can provide rudimentary sleep monitoring with little cost. However, a problem with sleep diaries is that they are reliant on an accurate recollection by the user of their sleep, which is generally not possible. As such, these sleep diaries are often unable to accurately monitor a quality of the user's sleep.

Sleep laboratories have also been used to monitor a user's sleep, where electrodes are placed on the user to automatically monitor the user's sleep. A problem with such sleep laboratories is that they require the user to sleep in an unknown area (i.e. at the sleep laboratory), and are generally complex and expensive. Such sleep laboratories and are thus often limited to use in medical diagnosis of serious sleep disorders.

More recently, portable devices such as smartphones and smart bracelets have been used to automatically monitor a user's sleep. In particular, a device is placed adjacent to, or worn by, the user during sleep. These device monitors movement of the user, which is then used to determine a sleep cycle of the user. A problem with such sleep monitoring systems of the prior art is that they are reliant on the user wearing the device or placing the device on the bed, or are generally not accurate.

Accordingly, there is a need for an improved sleep monitoring system and method.

It will be clearly understood that, if a prior art publication is referred to herein, this reference does not constitute an admission that the publication forms part of the common general knowledge in the art in Australia or in any other country.

### SUMMARY OF INVENTION

The present invention is directed to a system and method for sleep monitoring, which may at least partially overcome at least one of the abovementioned disadvantages or provide the consumer with a useful or commercial choice.

With the foregoing in view, the present invention in one form, resides broadly in a method of monitoring a user's sleep, the method including: monitoring, using one or more sensors of a bed, biological signal data of a user; determining, by a processor, a sleep phase of the user according to the biological signal data; and providing, on a data interface, a sleep cycle report, determined at least partially according to the sleep phase of the user.

The sleep phase of the user can include a deep sleep phase. The biological signal data can include at least one of movement data, heartbeat data, temperature data, and breathing rate data.

According to certain embodiments, a pressure of the bed is automatically adjusted according to the determined sleep phase. For example, the pressure can be increased when entering a ‘deep sleep’ sleep phase. As such, certain embodiments of the present invention can be used to actively advance the quality of the user's sleep.

The pressure of the bed can be automatically adjusted by adjusting an amount of air in a bladder of the bed. The skilled addressee will, however, readily appreciate that various other means can be employed to adjust the pressure of the bed.

According to certain embodiments, the method further comprises: adjusting a pressure of the bed to a first pressure; subsequent to adjusting the pressure of the bed, determining, by the processor, a further sleep phase of the user; and adjusting the pressure of the bed to a second pressure according to the further sleep phase and the first pressure.

According to some embodiments, the method further comprises: determining an awakening time according to the sleep phase; waking the user at the awakening time.

Waking the user can, for example, comprise activating an alarm, or adjusting a pressure of the bed. In particular, a pressure of the bed can be increased prior to activating an alarm.

According to certain embodiments, providing the sleep cycle report comprises displaying the sleep cycle report on a display screen. The display screen can be a display screen associated with the bed, a display screen of a smart device, or any other suitable display screen.

Providing the sleep cycle report can comprise sending, on a data interface, the sleep cycle report to a smart device for display on said smart device. For example, the sleep cycle report can be sent by a wireless local area network, or by near field communication (NFC).

According to some embodiments, providing the sleep cycle report comprises displaying a graph representing sleep cycle data.

According to certain embodiments, the method further comprises: receiving, on a data interface, metadata from a user of the bed; and associating the metadata with at least part of the sleep cycle report. The metadata can comprise at least one of: the user's intake of alcohol, the user's stress level, the user's activity level, the user's overall health; or the user's pain.

In another form, the present invention resides broadly in a bed system for monitoring a user's sleep, the bed system including: a bed including one or more sensors, for monitoring one or more biological signals of a user; a processor, coupled to the one or more sensors, for determining a sleep phase of the user according to the one or more biological signals; and a data interface, coupled to the processor, for providing a sleep cycle report, determined at least partially according to the sleep phase of the user.

The processor can be wirelessly coupled to the one or more sensors. The bed can include a pressure control system, coupled to the processor, for adjusting a pressure of the bed.

The bed system can further comprise a display screen, for displaying the sleep cycle report.

According to certain embodiments, the bed system further comprises a user interface, for receiving metadata from a user of the bed, wherein the metadata is associated with at least part of the sleep cycle report. The user interface can, for example, be displayed on a smart phone coupled to the processor. The metadata can comprise at least one of: the user's intake of alcohol, the user's stress level, the user's activity level, the user's overall health; or the user's pain.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS

Various embodiments of the invention will be described with reference to the following drawings, in which:

FIG. 1 illustrates a perspective view of a bed, according to an embodiment of the present invention;

FIG. 2 illustrates an elevation view of the bed of FIG. 1;

FIG. 3 illustrates a method of monitoring sleep of a user, according to an embodiment of the present invention;

FIG. 4 illustrates a method of sleep analysis, according to an embodiment of the present invention;

FIG. 5 illustrates a method of adjusting a bed, such as the bed of FIG. 1, according to an embodiment of the present invention;

FIG. 6 illustrates a method of sleep monitoring, according to an embodiment of the present invention;

FIG. 7 illustrates a method of controlling a bed, according to an embodiment of the present invention; and

FIG. 8 illustrates a bedding system, according to an embodiment of the present invention

Preferred features, embodiments and variations of the invention may be discerned from the following Description of Embodiments which provides sufficient information for those skilled in the art to perform the invention. The Description of Embodiments is not to be regarded as limiting the scope of the preceding Summary of Invention in any way.

### DESCRIPTION OF EMBODIMENTS

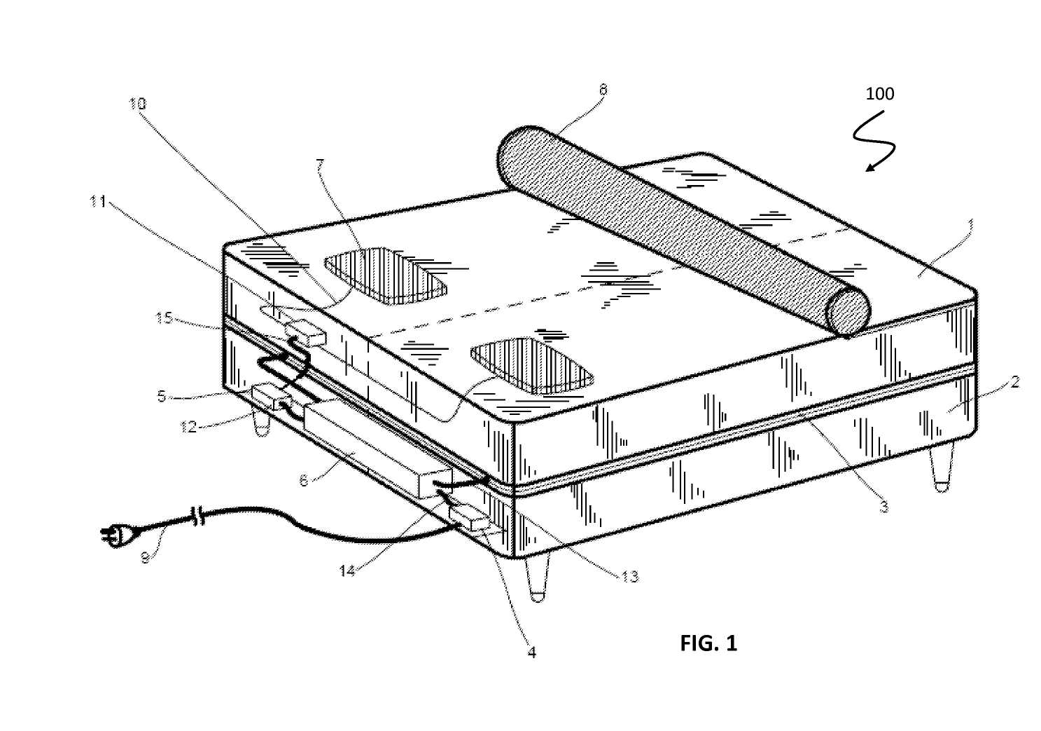

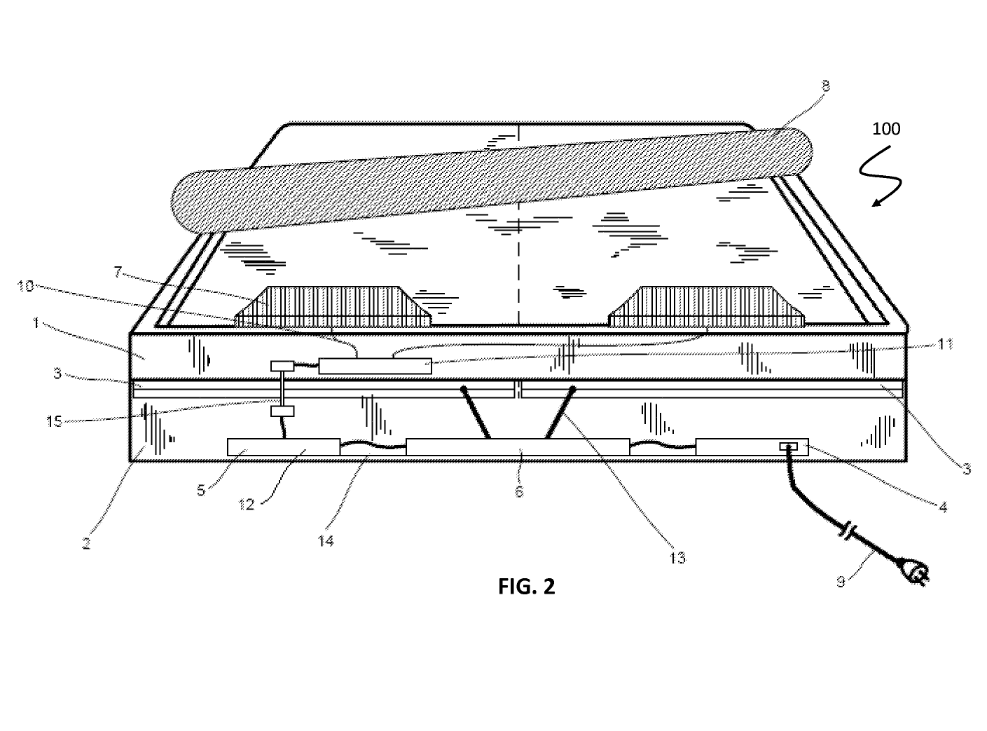

FIG. 1 illustrates a perspective view of a bed 100, according to an embodiment of the present invention. FIG. 2 illustrates an elevation view of the bed 100.

The bed 100 comprises an ensemble including a mattress 1 and base 2. The base 2 includes a pressure control system 6, a control board 5, a power system 4 and a wireless connection 12. The mattress 1 includes biological signal monitors 7 and a biological signal monitoring system 11.

The power system 4 provides power to all components within the base 2 and the mattress 1. The power system 4 connects externally to a power source via a power cable 9, as is well known in the art.

The systems within the base 2 are connected to and interface with one another via a series of cabling 14 capable of transmitting both power and data. Information from the biological signal monitoring system 11 is transferred to the control board 5 via cabling 15. The cabling 15 is also capable of transmitting both power and data.

A pressure system 3 is controlled by the pressure control system 6. The pressure control system 6 is connected to cabling 13 which enables the pressure control system 6 to perform pressure adjustment. In the case of an air bed system, the pressure control system 6 can include a pump and tubing to provide determined volumes of air to the pressure system 3. The pressure system 3 can in such case comprise a bladder the can be filled with volumes of air to adjust the pressure of the bed.

The mattress 1 encompasses ordinary mattress features including a mattress topper 8 to provide for increased comfort, support features and edging to aid with the rigidity of the mattress 1. The biological signal monitors 7 are situated below the mattress topper 8, to record the biological signals of the user in an effective manner. The biological signal monitors 7 are connected via cabling 10 to the biological signal monitoring system 11.

The power system 4, the pressure control system 6 and the biological signal monitoring system 11 are all located with the base 2. As such, the systems are kept out of view, and as such the bed 100 has an appearance similar to that of a regular bed. Furthermore, the systems are protected from external impact.

A remote control (not shown) may also be connected wirelessly or via wired connection to the control board 5. In such case, the remote control can contain controls for manual adjustment of the pressure, the engagement or disengagement of an automation mode, the engagement or disengagement of a waking mode and the engagement or disengagement of the connection to a smart device.

In use, the user's biological signals are monitored using the biological signal monitors 7 and the biological signal monitoring system 11, in which a sleep phase of the user is determined. In particular, the biological signal monitoring system 11 computes variation indices representing variation of the biological signals, which can in turn be used to generate thresholds for determining a sleep phase of the user.

A sleep cycle report including the biological signals, sleep phases, sleep cycles, or a measure of quality of a user's sleep, for example, can be presented to a user on a smart device (not shown) connected to the bed 100. Typically a single sleep cycle report is generated for a user per night.

During monitoring of the biological signals, the pressure control system 6 can be used to inflate or deflate air bladders of the bed to achieve a desired pressure according to the sleep phases. The bladders are generally surrounded by a soft border and encased by mattress ticking.

According to certain embodiments, the electric pump includes a remote control (tethered or wireless) which allows a user to inflate or deflate the bladder while awake. In such case, the manually operated remote control electronically communicates with the pressure control system 6.

The pressure control system 6 can monitor the pressure through various techniques and inflate or deflate the air bladders as necessary to ensure maintenance of the desired pressure.

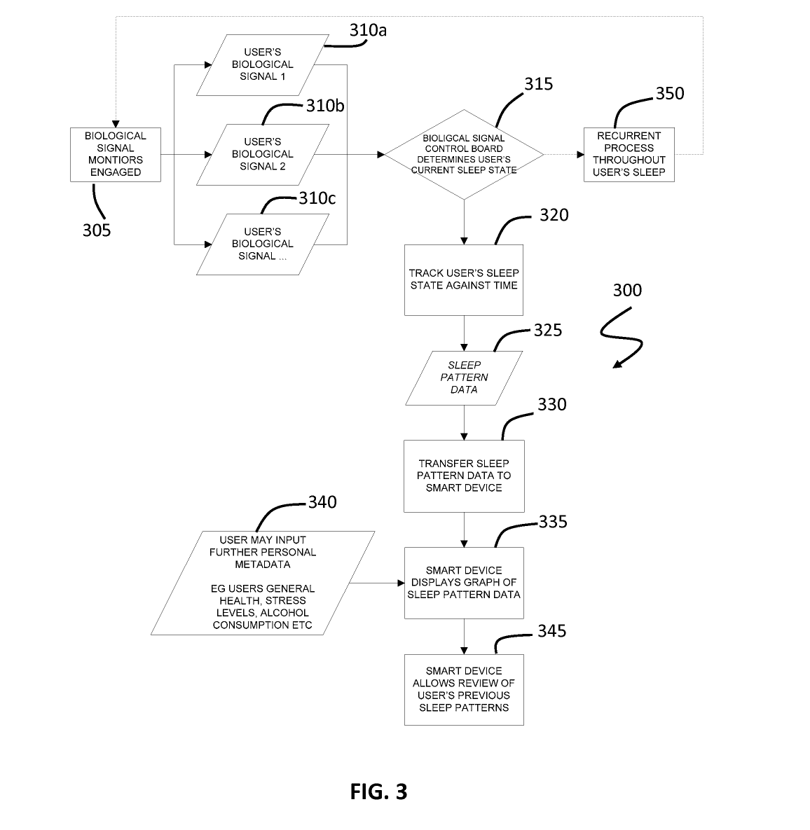

FIG. 3 illustrates a method 300 of monitoring sleep of a user, according to an embodiment of the present invention.

At block 305, one or more biological signal monitors are engaged. Examples of biological signal monitors include movement sensors, heartbeat sensors, temperature sensors, and breathing rate sensors. As illustrated by blocks 310a, 310b and 310c, the biological signal monitors can monitor a plurality of biological signals.

At block 315, a sleep phase of the user is determined. The sleep phase can be determined according to data of the biological signal monitors. In particular, the data of the biological signal monitors can be compared with predetermined or dynamic thresholds in order to determine the sleep phase. At block 320, the sleep phase of the user is logged against the time in which the sleep phase was determined. As a result, sleep pattern data (also referred to as sleep cycle data) is recorded, as illustrated by block 325.

At block 330, the sleep pattern data is transferred to a smart device, such as a smartphone, a tablet computer or the like. At step 335, the sleep pattern data, or a derivative thereof is displayed on the smart device. For example, a curve of the sleep pattern data over time, or a score/index can be presented.

As illustrated by block 340, the user can input personal metadata, such as a user's general health, stress levels, alcohol consumption, and the like. Such information can be provided to the smart device, and displayed in association with the sleep pattern data in step 335.

At block 345, the smart device enables the user to review previous sleep patterns. This enables a user to determine, for example, an impact of behaviour, such as alcohol consumption, on sleep patterns, with a view of adjusting such behaviour.

As illustrated by block 350, monitoring a user's biological signals and determining a user's sleep state are performed continuously throughout a user's sleep. As such, the sleep pattern data illustrated in block 325 can include sleep pattern data from several intervals, including an entire night.

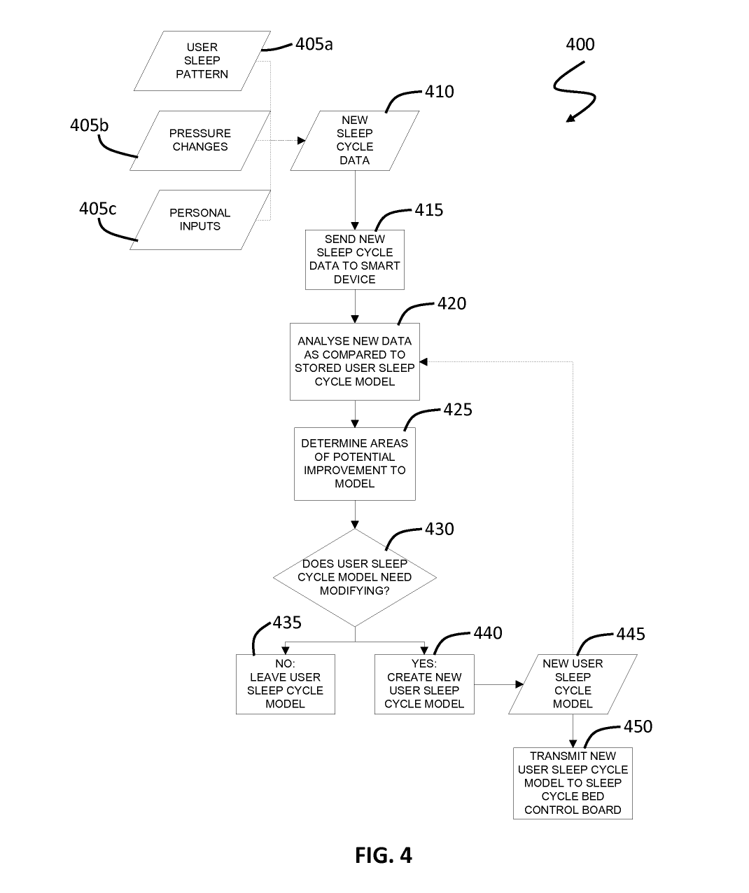

FIG. 4 illustrates a method 400 of sleep analysis, according to an embodiment of the present invention.

The method 400 can include as input user sleep pattern data, pressure data and/or personal inputs, as illustrated by blocks 405a, 405b, 405c. The inputs are used to generate new sleep cycle data as illustrated by block 410.

At block 415, the new sleep cycle data is sent to a smart device, such as a smart phone or tablet computer, and at block 420 the new sleep cycle data is analysed. Analysis of the new sleep cycle data includes a comparison of the new sleep cycle data with an existing sleep cycle model of the user.

At block 425, areas of potential improvement to the sleep cycle model of the user are determined. Such improvements can include, for example, improved compensation for pressure changes, or the like.

At block 430, it is determined if the existing sleep cycle model needs modification. Such step can, for example, include determining if the new sleep cycle data deviates from the sleep cycle model more than a threshold value. If not, the method concludes at block 435, and the sleep cycle model is left unchanged.

If, however, it is determined that the sleep cycle model needs modification, a new sleep cycle model is generated in block 440, as illustrated by the new sleep cycle model in block 445. The new sleep cycle model is then transmitted to a bed, such as the bed 100, for future use by the bed in block 450. The new sleep cycle model is further provided to block 420, where it can be compared with further new sleep cycle data. As such, the method 400 is able to continuously improve.

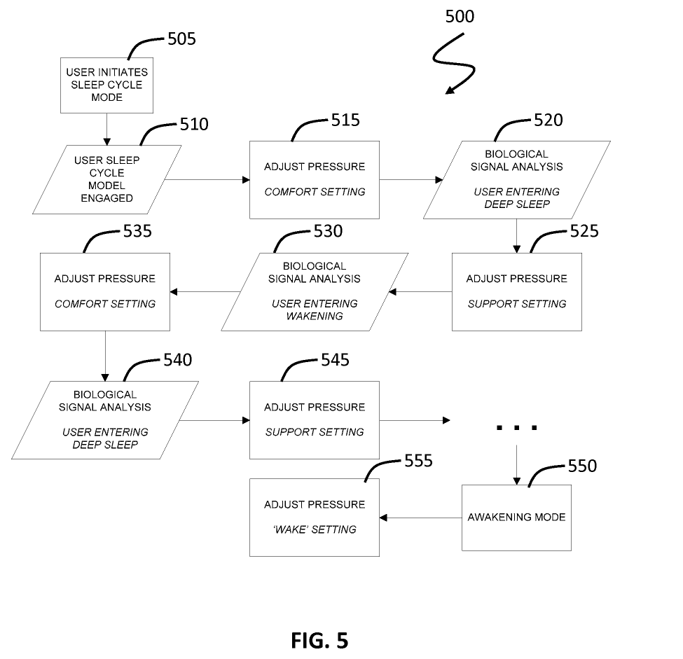

FIG. 5 illustrates a method 500 of adjusting a bed, such as the bed 100, according to an embodiment of the present invention.

At block 505 the user initiates a sleep cycle model, for example by selecting a button of the bed, and at block 510 the sleep cycle model is engaged in response thereto. At block 515, a pressure of the bed is adjusted according to a comfort setting according to the user sleep cycle model. The comfort setting can, for example, be a baseline firmness setting that the user has selected, and can help the user enter deep sleep.

At block 520, biological signal analysis is used to determine a sleep phase of the user, and in particular that the user is entering deep sleep. At block 525, the pressure of the bed is adjusted according to a support setting according to the user sleep cycle model. The support setting can, for example, help the user stay in a deep sleep, or otherwise be beneficial for deep sleep.

At block 530, biological signal analysis is used to determine a further sleep cycle of the user, and in particular that the user is entering a wakening phase. At block 535, the pressure of the bed is adjusted according to the comfort setting, to help the user re-enter deep sleep.

At block 540, biological signal analysis is used to again determine that the user is entering deep sleep, and at block 545, the pressure of the bed is adjusted according to the support setting. As will be readily appreciated by the skilled addressee, the user may go in and out of deep sleep and number of times during a night, and as such, the pressure of the bed can be adjusted any suitable number of times.

At block 550, an awakening mode is set. Awakening mode can be set according to an alarm clock and a sleep cycle of the user. For example, the awakening mode can be entered within a predetermined time period when the user enters a wakening mode. At block 555, the pressure of the bed is adjusted according to a wakening setting. The wakening setting can, for example, comprise a firm setting that encourages the user to wake up rather than re-enter a deep sleep.

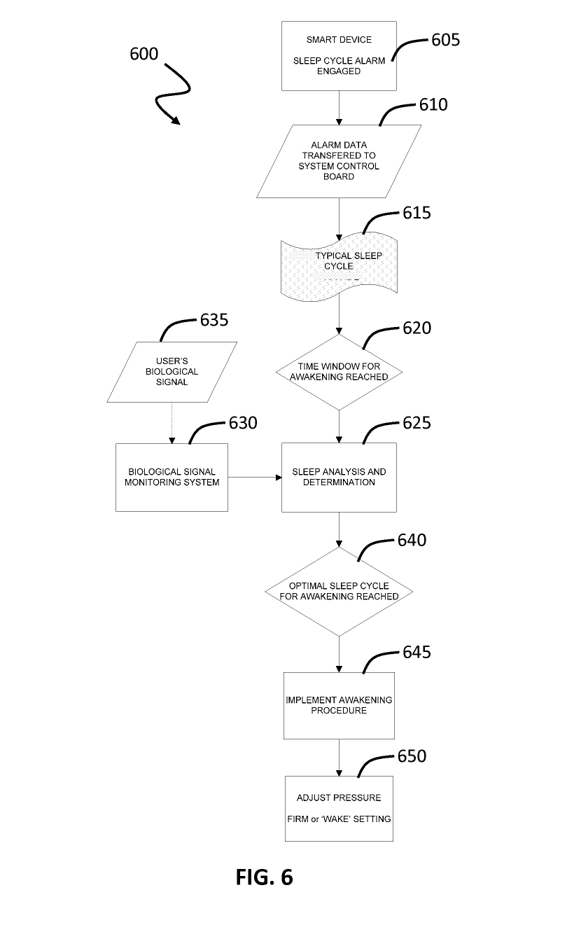

FIG. 6 illustrates a method 600 of sleep monitoring, according to an embodiment of the present invention.

At block 605, a sleep cycle alarm is engaged on a smart device. The sleep cycle alarm can be engaged in a manner similar to a standard alarm, for example through manual selection of a wake-up time. The alarm data is then transferred from the smart device to the bed in block 610.

At block 615, the user transitions through different sleep cycles, to which the bed adapts, for example as illustrated with reference to FIG. 5. This is repeated until a time window for awakening is reached, as illustrated with reference to block 620.

The time window for awakening can be determined according to a desired wake-up time, and a wake-up time window. The wake up time window can be around the desired wake-up time, for example entirely before the desired wake-up time, or partly before and partly after the desired wake up time. As such, the time window can provide certainty regarding the timeframe when a user is woken.

Subsequent to determining that the time window for awakening has been reached, sleep analysis is performed as illustrated with reference to block 625. In particular, sleep analysis is performed according to the user's biological signals as illustrated in block 635, and a biological signal monitoring system as illustrated in block 630.

At block 640, an optimal sleep cycle for awakening is reached, for example by the user leaving deep sleep, upon which an awakening procedure is implemented in block 645. The awakening procedure can, for example, include an audible alarm and/or lights, and as illustrated with reference to block 650, a pressure of the bed can be adjusted to a firm or wake setting, to assist the user in waking.

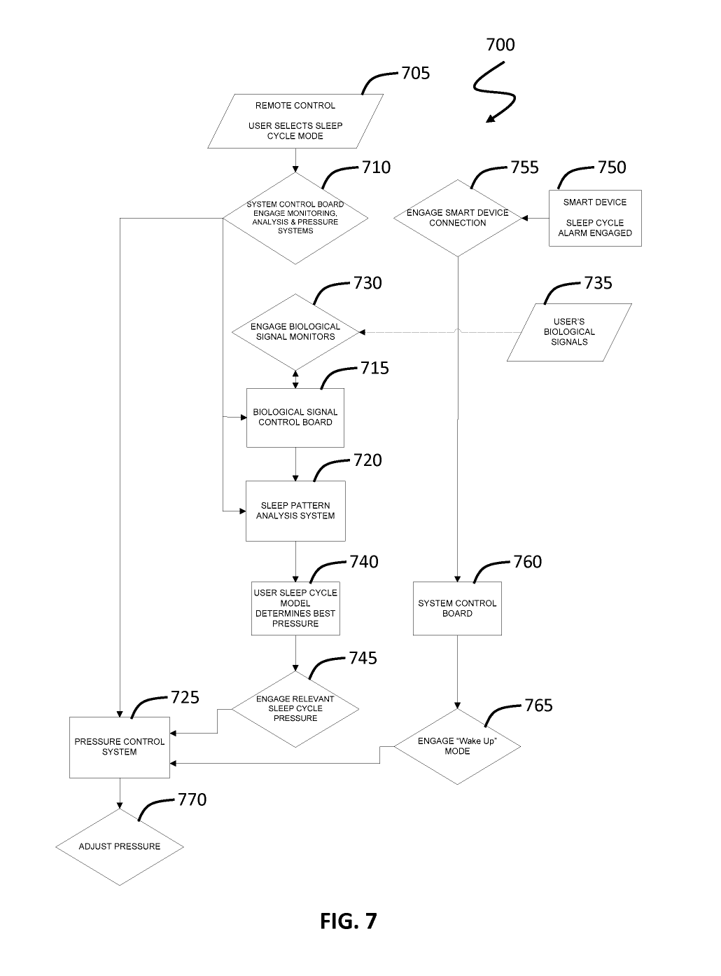

FIG. 7 illustrates a method 700 of controlling a bed, according to an embodiment of the present invention.

At block 705, a user selects a sleep cycle model using a remote control of the bed. As discussed earlier, the selected sleep cycle mode can, for example, comprise a baseline comfort setting of the users choosing.

At block 710, the system control board engages monitoring, analysis and pressure systems by a biological signal control board as illustrated in block 715, a sleep pattern analysis system as illustrated in block 720 and a pressure control system as illustrated in block 725.

The biological control board engages biological signal monitors, as illustrated by block 730, which capture biological signals of a user, illustrated by block 735.

As illustrated by block 740, a suitable pressure of the bed is determined according to the sleep cycle model, and the pressure of the bed is set to the suitable pressure in 745.

A smart device, such as a smart phone or tablet computer, illustrated by block 750, is engaged with the bed, as illustrated by block 755. In particular, the smart device is engaged with a system control board, illustrated by block 760, which can engage a wake up mode, as discussed earlier and as illustrated by block 765.

The pressure control system is can adjust a pressure of the bed, as illustrated by block 770, for example by pumping air in or out of a bladder of the bed.

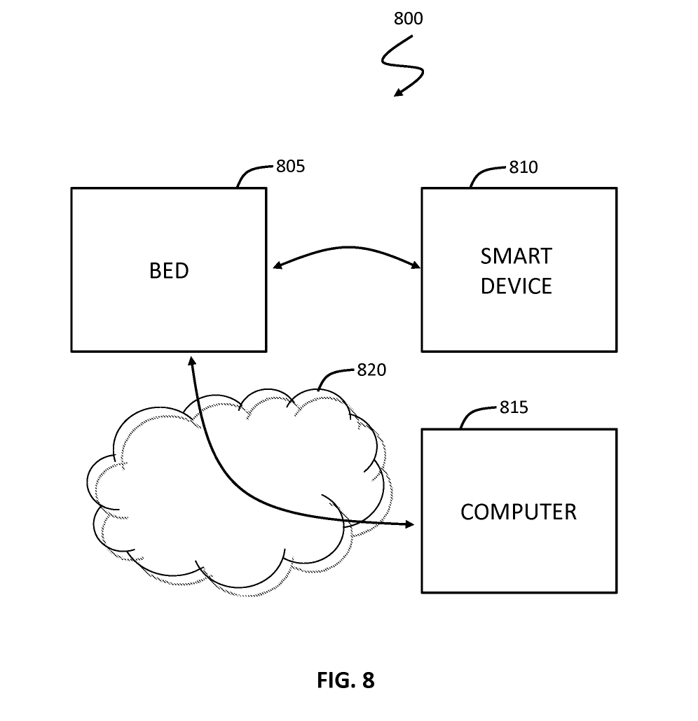

FIG. 8 illustrates a bedding system 800, according to an embodiment of the present invention.

The bedding system 800 includes a bed 805, a smart device 810 and a computer 815. The bed includes one or more sensors, for monitoring one or more biological signals of a user. As discussed above, the one or more biological signals can include movement, heart rate, temperature or the like.

The bedding system 800 further includes a processor (not shown), coupled to the one or more sensors, for determining a sleep phase of the user according to the one or more biological signals. The processor can form part of the bed 805, the smart device 810, the computer 815, or a combination thereof. A sleep cycle model can be used to determine the sleep phase of the user.

The bedding system 800 further includes a data interface (not shown), coupled to the processor, for providing a sleep cycle report, determined at least partially according to the sleep phase of the user. In particular, the bed 805 includes a wireless interface, such as a near field communication (NFC) interface or a wireless local area network (WLAN) interface, for communicating with the smart device 810 and the computer 815. In such case, the bed 805 can communication directly with the smart device 810, and indirectly with the computer 815, for example by a communications network 820, such as the Internet.

The sleep cycle report can, for example, include the varying patterns or stages of human sleep, presented graphically on a timeline, or comprise one or more sleep indices that are presented textually.

According to some embodiments, the present invention enables a bed to record sleep data of a user for display on a smart device. The sleep data and the representation of patterns provide users reference material which can be used to improve the user's sleep in the future. In some embodiments, the user is able to provide metadata in relation to any individual sleep that will allow for greater efficiency in the categorization of a sleep pattern and also greater context for any review of past patterns. Metadata in this instance includes characteristics of a user's life, recent past, general health or other matter that may have an influence on a user's general sleep. For example a user's intake of alcohol, current stresses level, recent activity level, overall health or any current pain. As such, a user may determine that alcohol consumption is a significant factor in determining sleep quality, and may regulate alcohol intake accordingly.

According to certain embodiments, the present invention provides an improved bed in which a user's biological signals are used to adjust pressure automatically during the user's sleep. In such case, active control is not required throughout the user's sleep, as biological signals are used to determine the user's sleep cycle and automatically adjust the bed to achieve the appropriate pressure. As such, embodiments of the present invention can improve the quality of the user's sleep by regulating the periods and quality of deep sleep.

Certain embodiments of the present invention provide a bedding system that provides continual ‘learning’ and adaptation of the pressure automation. In such case, sleep data can be compared and analysed against a standard automation model to determine if the appropriate pressure is being delivered to maximize the quality of the user's sleep.

Certain embodiments of the present invention enables a bed to utilize wired and or wireless technology to interface with a smart device to control the mechanical and electrical functions of a bed for the purpose of actively and automatically maximizing the quality of user's sleep. The connectivity will be provided through any form of wired or wireless transmission that allows the movement of data in both directions between the bed and a smart device.

Finally, certain embodiments of the present invention enable an integrated process specifically for the purpose of waking a user. This can be achieved through the nomination of a time window by the user prior to using the bed, upon which an awakening time is determined in that time window according to the user's sleep pattern. For example, a pressure of the bed can be adjusted to influence how the user is woken.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. A method of monitoring a user's sleep, the method comprising:
monitoring, using one or more sensors of a bed, biological signal data of a user;
determining, by a processor, a sleep phase of the user according to the biological signal data; and
providing, on a data interface, a sleep cycle report, determined at least partially according to the sleep phase of the user.

2. The method of claim 1, wherein the sleep phase of the user includes a deep sleep phase.

3. The method of claim 1, wherein the biological signal data includes at least one of movement data, heartbeat data, temperature data, and breathing rate data.

4. The method of claim 1, further comprising automatically adjusting a pressure of the bed according to the determined sleep phase.

5. The method of claim 4, wherein automatically adjusting a pressure of the bed comprises adjusting an amount of air in a bladder of the bed.

6. The method of claim 1, further comprising:
adjusting a pressure of the bed to a first pressure;
subsequent to adjusting the pressure of the bed, determining, by the processor, a further sleep phase of the user; and
adjusting the pressure of the bed to a second pressure according to the further sleep phase and the first pressure.

7. The method of claim 1, further comprising:
determining an awakening time according to the sleep phase; and
waking the user at the awakening time.

8. The method of claim 7, wherein waking the user comprises activating an alarm.

9. The method of claim 8, wherein waking the user comprises adjusting a pressure of the bed.

10. The method of claim 1, wherein providing the sleep cycle report comprises displaying the sleep cycle report on a display screen.

11. The method of claim 10, wherein providing the sleep cycle report comprises sending, on a data interface, the sleep cycle report to a smart device for display on said smart device.

12. The method of claim 1, wherein providing the sleep cycle report comprises displaying a graph representing sleep cycle data.

13. The method of claim 1, further comprising:
receiving, on a data interface, metadata from a user of the bed; and
associating the metadata with at least part of the sleep cycle report.

14. The method of claim 13, wherein the metadata comprises at least one of: the user's intake of alcohol, the user's stress level, the user's activity level, the user's overall health; or the user's pain.

15. A bed system for monitoring a user's sleep, the bed system comprising:
a bed comprising one or more sensors, for monitoring one or more biological signals of a user;
a processor, coupled to the one or more sensors, for determining a sleep phase of the user according to the one or more biological signals; and
a data interface, coupled to the processor, for providing a sleep cycle report, determined at least partially according to the sleep phase of the user.

16. The bed system of claim 15, wherein the processor is wirelessly coupled to the one or more sensors.

17. The bed system of claim 15, wherein the bed includes a pressure control system, coupled to the processor, for adjusting a pressure of the bed.

18. The bed system of claim 15, further comprising a display screen, for displaying the sleep cycle report.

19. The bed system of claim 15, further comprising a user interface, for receiving metadata from a user of the bed, wherein the metadata is associated with at least part of the sleep cycle report.

20. The bed system of claim 15, wherein the metadata comprises at least one of: the user's intake of alcohol, the user's stress level, the user's activity level, the user's overall health; or the user's pain.
